(19) United States
(12) Patent Application Publication
(10) Pub. No.: US 2007/0059682 A1
(43) Pub. Date: Mar. 15, 2007

(54) Method to increase specificity and/or accuracy of lateral flow immunoassays

(75) Inventors: Franz Aberl, Princeton, NJ (US); Marcus Schreibenzuber, Muenchen (DE); Robert P. Sambursky, Bradentown, FL (US); Robert W. Vandine, Montoursville, PA (US); Jose S. Sambursky, Johnson City, NY (US)

Correspondence Address: ROTHWELL, FIGG, ERNST & MANBECK, P.C., 1425 K STREET, N.W., SUITE 800, WASHINGTON, DC, 20005, US

(73) Assignee: Rapid Pathogen Screening Inc., Johnson City, NY (US)

(21) Appl. No.: 11/224,298

(22) Filed: Sep. 13, 2005

(51) Int. Cl. C12Q 1/70; G01N 33/554; G01N 33/558; G01N 33/569

(52) U.S. Cl. 435/5; 435/7.32; 436/514

(57) ABSTRACT

The present invention relates to a method for detecting an analyte in a sample, wherein the sample to be analyzed is applied to a chromatographic carrier. After separating from an interfering substance which may be present in the sample, the analyte of interest is detected on the carrier by means of an immunological assay. Further, a test strip for carrying out the method of the invention is provided. The invention further relates to a method for reducing interference in a method for detecting an analyte on a chromatographic carrier.

### TECHNICAL FIELD OF THE INVENTION

The present invention relates to a method for detecting an analyte in a sample, wherein the sample to be analyzed is applied to a chromatographic carrier. After separating from an interfering substance which may be present in the sample, the analyte of interest is detected on the carrier by means of an immunological assay. Further, a test strip for carrying out the method of the invention is provided. The invention further relates to a method for reducing interference in a method for detecting an analyte on a chromatographic carrier.

### BACKGROUND OF THE INVENTION

Lateral flow immunoassays are a subset of antibody-based immunoassays combining various reagents and process steps in one assay strip, thus providing a sensitive and rapid means for the detection of target molecules. Lateral flow immunoassays are available for a wide area of target analytes and can be designed for sandwich or competitive test principles. Generally high molecular weight analytes with several epitopes are analyzed in a sandwich format whereas small molecules representing only one epitope are detected by means of a competitive assay. The first tests were made for human chorionic gonadotropin (hCG). Today there are commercially available tests for monitoring ovulation, detecting infectious disease organisms, analyzing drugs of abuse and measuring other analytes important to human physiology. Products have also been introduced for veterinary testing, environmental testing and product monitoring.

U.S. Pat. No. 5,714,341 discloses a lateral flow immunoassay for HIV specific antibodies in saliva samples. The saliva sample is diluted in a sample buffer and the lateral flow immunoassay is dipped into the diluted saliva sample. The disclosure of this document is herein incorporated by reference.

German Patent DE 196 22 503 suggests to apply lateral flow immunoassays for the detection of illegal narcotics in saliva or sweat. The disclosure of this document is herein incorporated by reference.

U.S. patent application Ser. No. 11/052,748 discloses the use of lateral flow immunoassays for the diagnosis of conjunctivitis by analyzing an eye fluid sample. The disclosure of this document is herein incorporated by reference.

However, the growing use of antibody based immunoassays in recent years has required increased effort and investigation on minimizing interferences found in many samples. A typical problem is the occurrence of interfering substances, e.g. antibodies, in whole blood, serum and other human fluid samples. These interfering antibodies can be divided into auto-antibodies or rheumatoid factors (RF), heterophilic antibodies and human anti-mouse antibodies (HAMA).

Auto-antibodies or rheumatoid factors (RF) show anti-IgG activity and are predominantly composed of the IgM class. Most often they recognize the Fc region of the antigen bound IgG antibodies. Rheumatoid factor antibodies may also be of the IgG and IgA classes and have been observed reacting with antibodies of the IgM class. To further complicate this group of interfering antibodies, rheumatoid factors from one species may react with immunoglobulins of another species.

Heterophilic antibodies are one of many sources of interference in immunoassays. This often-misapplied term was historically used to refer to certain populations of antibodies in patient sera, which caused the aggregation of sheep red blood cells, and observed to be associated with Epstein-Barr virus (EBV) infections. In immunoassay development labs today, the term heterophile is frequently used to describe an interfering antibody (or other binding molecule) which has an unknown origin. These relatively common low affinity antibodies occur in approximately 1-5% of the healthy population and effectively compete with the analyte of interest, which may produce abnormally high or false positive immunoassay results.

Human anti-mouse antibodies (HAMA) are high affinity human anti-animal antibodies, which are directed against specific animal immunoglobulins. Human anti-mouse antibodies have been reported to give false positive results in sandwich immunoassays that utilize mouse monoclonal IgG. HAMA reactivity has been detected in approximately 9% of the normal population. The patient sample contains an antibody to mouse immunoglobulin due to the previous exposure to mouse antibodies. This can occur through diet or through exposure, or may be a direct result of monoclonal antibody therapy—a presently uncommon, but growing subset of the patient population. Actually, not all HAMAs are human anti-mouse antibodies. Many prove to be anti-rabbit, anti-dog, etc. Since immunoglobulins are highly conserved across species, it is not uncommon to see a patient with an antibody titer to immunoglobulins exhibiting cross-reactivity to mouse IgG.

All these interfering antibodies are capable of simulating an analyte of interest when body fluids are tested in an immunoassay. This interference can result in false positives, false negatives and all graduations in between these two extremes. Thus, it is the objective of the invention to provide a method for the detection of analytes in body fluids in the presence of interfering substances to increase specificity and/or accuracy of lateral flow immunoassays.

### SUMMARY OF THE INVENTION

In a first aspect, the present invention relates to a method for detecting an analyte in a sample , comprising the steps:
(a) applying the sample to a chromatographic carrier,
(b) capturing an interfering substance possibly present in the sample on the carrier and thereby separating the analyte from the interfering substance, and
(c) detecting the analyte separated from the interfering substance on the carrier.

In a further aspect, the invention relates to a test strip for detecting an analyte in a sample, comprising:
(a) an application zone for applying the sample,
(b) a reagent zone containing reagents for detecting the analyte,
(c) a capturing zone for separating an interfering substance from the sample,
(d) a detection zone for detecting the analyte, and
(e) optionally a waste zone.

In a further aspect, the invention relates to a method for reducing interference in a method for detecting an analyte on a chromatographic carrier, comprising the steps:
(a) applying a sample to the carrier,
(b) passing the sample over a capturing zone located on the carrier thereby separating the analyte from an interfering substance, and after step (b)
(c) passing the sample to a detection zone located on the carrier thereby detecting the analyte.

### DESCRIPTION OF THE DRAWINGS

FIG. 1 schematically shows a sandwich immunoassay strip which is typically used for the detection of microbial antigens in serum and other body fluids or pregnancy testing. The immunoassay strip consists of an application zone, a reagent zone comprising a labeled antibody specific to the analyte, a test line comprising a test line antibody specific to the analyte, and a waste zone. The drawings show a specific embodiment of an immunoassay strip design. Alternative embodiments may arrange sample application and reagent zone in a different way, e.g. the application zone may be positioned downstream to the reagent zone and/or reagents may be mobilized by an additional chromatograhic fluid.

FIG. 2 shows the lateral flow immunoassay strip with an analyte sandwich-complex. The sample fluid is flowing over the strip and the analyte is “sandwiched” between a labeled, non-immobilized antibody and an immobilized test line antibody. Both antibodies are specific to the analyte and may be mouse anti target antibodies—here indicated by an “M”.

FIG. 3 shows the presence of human anti-mouse antibodies (HAMA) leading to a positive signal in the absence of the analyte as a result of bridging the soluble and the immobilized analyte specific antibody. HAMA antibodies utilize mouse specific epitopes on the soluble, labeled antibody and the immobilized antibody, respectively.

FIG. 4 shows the introduction of a capturing zone into the immunoassay strip. The capturing zone is eliminating the interfering antibody from the sample and avoids false positive results.

FIG. 5 shows a sample analysis device in the form of a chromatographic test strip comprising a plurality of different strip materials building an absorbant pad (1), an application zone (2), a reagent zone (3), a capturing zone (4), a detection zone (5) and a waste zone (6). The strip materials are arranged on an adhesive plastic backing (7). The absorbant pad (1) is provided for adding an elution medium in order to faciliate the transfer of the sample to the detection zone (5).

### DETAILED DESCRIPTION OF PREFERRED EMBODIMENTS

The invention provides a sensitive and rapid method for the detection of analytes, e.g. pathogens and/or low-molecular-weight compounds, in samples which may contain interfering substances. The detection may comprise a direct detection of the analyte and/or the detection of antibodies against the analyte, which are present in the fluid sample to be tested. Preferably, the method comprises a parallel determination of a plurality of analytes. The pathogens are selected from viruses or microorganisms, such as bacteria or parasites. The low-molecular-weight compounds may comprise drug molecules. Interfering substances according to the invention are selected from antibodies, e.g. human-anti-mouse antibodies (HAMA), or compounds exhibiting structural similarity with the analyte, e.g. low-molecular-weight compounds.

The body fluid to be tested is preferably whole blood, serum or a body surface fluid. More preferably, the sample is selected from mucous membrane fluids or secretions from glands. Most preferably, the body fluid is an eye fluid, sweat or saliva. The sample required to perform an analysis is preferably about 0.1 μl to about 100 μl, more preferably about 0.2 μl to about 20 μl and most preferably about 0.5 μl to about 10 μl. The test results are generally provided within short time, preferably in a time period up to 20 minutes, more preferably up to 15 minutes. The invention may be performed by means of a test strip. Handling of the test strip does not necessitate additional laboratory equipment, further handling of reagents or instrumentation.

The analyte of interest is preferably a pathogen or a plurality of pathogens being detected by immune reaction with an immobilized binding molecule specific to the analyte. More preferably, the analyte is a pathogen or a plurality of pathogens associated with conjunctivitis, an inflammation of the eye which is often caused by an infection. Most preferably, the analyte is a pathogen selected from the group consisting of adenoviruses, herpesviruses, chlamydiae, cytomegaloviruses, pseudomonas, streptococci, haemophilus, staphylococci, amobae and combinations thereof.

Alternatively, the analyte of interest may be a low-molecular-weight compound. In a preferred embodiment, the analyte to be detected is a drug molecule such as heroin or methamphetamine.

The invention also comprises the detection of a plurality of pathogens or low molecular-weight compounds on a single chromatographic carrier. The sample analysis device may allow a simultaneous detection of a plurality of low molecular-weight compounds or pathogens, particularly of at least two, of at least three, of at least four or of at least five pathogens selected from the group consisting of adenoviruses, herpesviruses, chlamydiae, cytomegaloviruses, pseudomonas, streptococci, haemophilus, staphylococci, amobae and combinations thereof.

In the method of the invention the interfering substance is captured on the carrier and thereby separated from the analyte(s) of interest. Preferably, the capturing step comprises an immune reaction. In particular, the immune reaction comprises immobilizing the interfering substance with an immobilized capturing reagent specific to the the interfering substance. The interfering substance may be an antibody which interfers with the detection of the analyte, e.g. by reacting with detection reagents. For example, the antibody may be selected from human anti-mouse antibodies (HAMA), heterophilic antibodies, rheumatoid factors (RF) or any combination thereof. In an especially preferred embodiment, the interfering compound is a human anti-mouse antibody (HAMA). Interfering antibodies may be separated from the sample by reaction with a capturing reagent which specifically recognizes the interfering antibody, but does not react with the analyte or the detection reagent. For example, a human-anti mouse antibody (HAMA) may be separated from the analyte by an immune reaction with a monoclonal or a polyclonal mouse antibody.

In an alternative embodiment, the interfering substance may be a low-molecular-weight compound, e.g. a drug molecule, exhibiting structural similarity with the analyte. Especially preferred interfering substances are legal drugs like morphine, codeine or dihydrocodeine, all of which show high structural similarity with the illegal drug heroin and its metabolite 6-monoacetylmorphine. 6-monoacetylmorphine is measured with laboratory based instrumental methods to differentiate between legal and illegal use of opiates. Other preferred low-molecular-weight interfering substances according to the invention are amphetamine, Ecstasy or ephedrines, which exhibit interfering properties when analyzed in combination with the differently classified drug methamphetamine. Amphetamine or ephedrines are subcompounds in medications whereas methamphetamine is a classified illegal drug. Interfering drug analogues may be separated from the sample by reaction with a capturing reagent which specifically recognizes the interfering drug analogue, but does not react with the target analyte or the detection reagent.

In the method of the invention, the sample to be analyzed for the analyte of interest is applied to a chromatographic carrier. The carrier can be made of one single chromatographic material, or preferably several capillary active materials made of the same or different materials and fixed on a carrier backing. These materials are in close contact with each other so as to form a transport path along which a liquid driven by capillary forces flows from an application zone, passing a reagent zone and at least one capturing zone, towards one or more detection zone(s) and optionally a waste zone at the other end of the carrier. In an especially preferred embodiment, the carrier is a chromatographic test strip.

Preferably, the sample is directly applied to the carrier by dipping the carrier's application zone into the sample. Alternatively, application of the sample to the carrier may be carried out by collecting the sample with a dry or wetted wiping element from which the sample can be transferred, optionally after moistening, to the carrier's application zone. Usually, the wiping element is sterile and may be dry or pretreated with a fluid before the collection step. Materials suitable for wiping elements according to the invention may comprise synthetic materials, woven fabrics or fibrous webs. Such wiping elements are described in German Patents DE 44 39 429 and DE 196 22 503, which are hereby incorporated by reference.

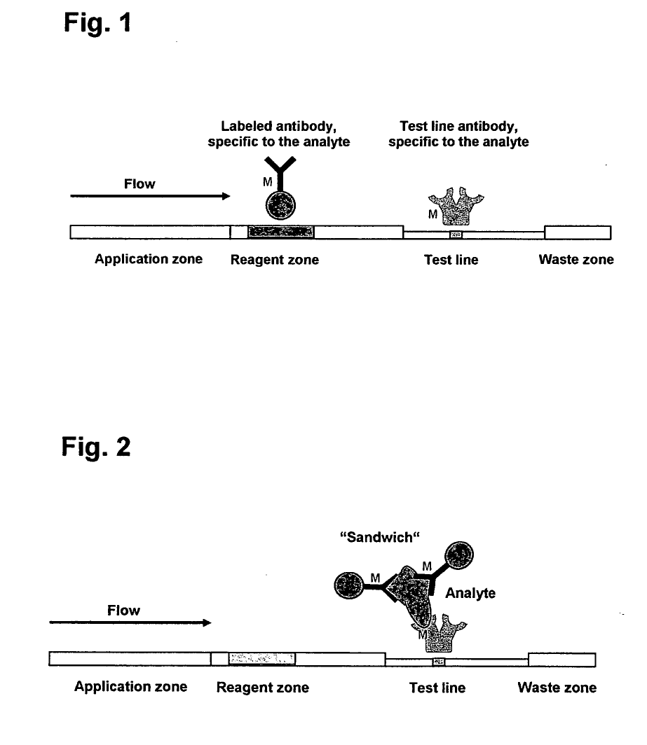

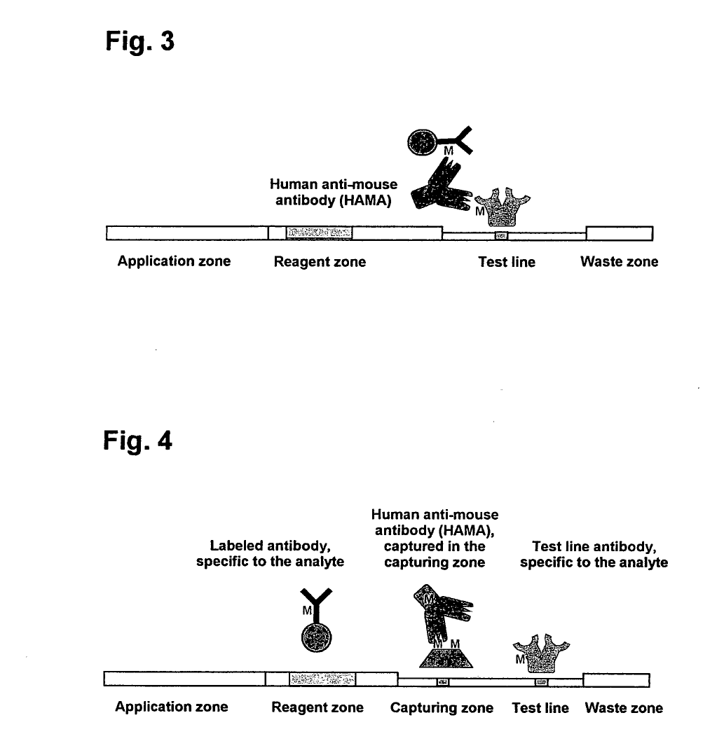

Depending on the type of detection method, different reagents are present in the carrier's reagent zone, which is preferably located between the application zone and the detection zone. In a sandwich immunoassay, it is preferred to have a labeled, non-immobilized reagent specific to the analyte in the reagent zone. The reagent forms a complex with the analyte which is bound to an immobilized analyte binding partner at a test line in the detection zone. In a competitive immunoassay, the reagent zone preferably contains a labeled, non-immobilized analyte analogue which competes with the analyte for the immobilized analyte binding partner in the detection zone. The analyte binding partners in the reagent zone and in the detection zone are preferably monoclonal, polyclonal or recombinant antibodies or fragments of antibodies capable of binding to a pathogen or a low-molecular-weight compound. On the other hand, the reagents may also be antigens capable of binding to antibodies against a pathogen or a low-molecular-weight compound. Other types of binding partners are biological compounds like receptors or RNA- or DNA-macromolecules or synthetic bioorganic macromolecules such as aptamers or artificial receptors. As an interfering substance, e.g. a human anti-mouse antibody (HAMA), is also capable of forming a complex with the labeled, non-immobilized reagent of the reagent zone and the immobilized analyte binding partner of the detection zone, thus indicating a positive test result in the immunoassay (FIG. 1c), the carrier further comprises at least one capturing zone. Each capturing zone contains an immobilized capturing reagent specifically binding to a certain interfering substance, thereby immobilizing the interfering substance(s) in the capturing zone(s). As the capturing zone is separated from the detection zone by space, and the sample starts to migrate over the reagent zone and the capturing zone before reaching the carrier's detection zone, the method allows a separation of the interfering substance(s) from the analyte(s) of interest (FIG. 1d). Preferably, the capturing zone is located between the reagent zone and the detection zone. However, the capturing zone may also be located between the application zone and the reagent zone.

Detection of the analyte(s) being separated from the interfering substance(s) is achieved by at least one detection zone present on the carrier, the detection zone(s) comprising the immobilized binding molecule(s) specific to the analyte(s). The binding molecule immobilizes the labeled analyte or the labeled analyte-analogue by immune reaction in the detection zone, thus building up a visible test line in the detection zone during the immunoassay process (FIG. 1b). Preferably, the label is an optically detectable label. Forming a complex at the test line concentrates and immobilizes the label and the test line gets visible for the bare eye, indicating a positive test result. Particularly preferred are direct labels, and more particularly gold labels which can be best recognized by the bare eye. Additionally, an electronically read out device (e.g. on the basis of a photometrical, acoustic, impedimetrical, potentiometric and/or amperometric transducer) can be used to obtain more precise results and a semi-quantification of the analyte. Other labels may be latex, fluorophores or phosphorophores.

The present invention also discloses a test strip for the performance of the method described above. In a preferred embodiment, the test strip comprises an application zone for applying the sample, a reagent zone containing reagents for detecting the analyte, a capturing zone for separating the interfering substance from the sample, a detection zone for detecting the analyte, and optionally a waste zone. The detection zone may comprise further sections for the detection of other analytes and at least one control section, e.g. a control line comprising an immobilized specific binding partner of an indicator substance indicating the functionality of the test strip.

The invention further relates to a method for reducing interference in a method for detecting an analyte on a chromatographic carrier. After applying a sample to the carrier, reduction of interference can be realized by passing the sample containing an interfering substance, e.g. an antibody or a low-molecular-weight compound, over a capturing zone located on the carrier, thereby separating the analyte(s) from the interfering substance, and subsequently passing the sample to a detection zone also located on the carrier for detecting the analyte.

### EXAMPLE

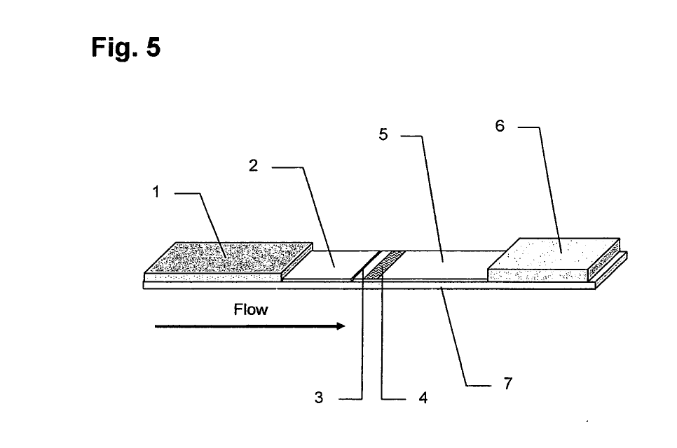

In a preferred embodiment of the invention a sample analysis device in the form of a chromatographic test strip as shown in FIG. 5 is provided. comprising is provided. The chromatograhic test strip comprises a plurality of different strip materials arranged on an adhesive plastic backing (7), the different strip materials building an absorbant pad (1), an application zone (2), a reagent zone (3), a capturing zone (4), a detection zone (5) and a waste zone (6). The absorbant pad (1) is provided for adding an elution medium in order to faciliate the transfer of a sample to be analyzed from the application zone (2) to the detection zone (5). The reagent zone (3) of the chromatographic test strip comprises a non-immobilized reagent specific to the analyte, whereas the detection zone (5) comprises an immobilized reagent specific to the analyte. In the capturing zone (4) of the chromatographic test strip a human anti-mouse antibody (HAMA) capturing reagent is immobilized. After applying a patient sample to the application zone (2), the sample and the non-immobilized reagent for detecting the analyte will pass the capturing zone (4) during the elution process. Human anti-mouse antibodies (HAMA) possibly present in the patient samples will be captured in this zone in order to prevent false positive signals within the detection zone (5) of the test strip resulting from unspecific binding of HAMA to the detection antibodies.

Effectivity of preventing false positive signals due to human anti-mouse antibodies (HAMA) in patient samples was proofed by applying 5 μl HAMA-positive human blood plasma to the above-mentioned chromatographic test strip. Three different plasma samples with concentrations of 100 ng/ml, 1494 ng/ml and 161 ng/ml of HAMA were used. In the capturing zone (4) of the test strip purified non specific murine IgG was immobilized, which was applied at a concentration of 3 mg/ml and a dispensing rate of 0.3 μl/mm. The test strip was designed for detecting virus antigen in human body fluids using monoclonal mouse antibodies both as a capturing reagent in the capturing zone (4) and as an immobilized detection antibody in the detection zone (5). As the non-immobilized detection reagent located in the reagent zone (3) gold-labeled monoclonal mouse antibodies were used.

Performing a comparative test on a chromatographic test strip not comprising a capturing zone (4) and applying HAMA-positive plasma thereto led to clearly false positive signals within the detection zone (5). When performing a test according to the invention on a test strip comprising the capturing zone (4), binding of the non-immobilized gold-labeled antibodies to the capturing zone (4) due to formation of a complex with human anti-mouse antibodies (HAMA) was observed. However, no false positive signals were detected. In addition, no influence on true positive signals could be observed when confirmed virus-positive samples were tested.

1. A method for detecting an analyte in a sample which possibly also contains an interfering substance, comprising the steps:
(a) applying the sample to a chromatographic carrier,
(b) separating the analyte from the interfering substance, by capturing on the carrier, an interfering substance possibly present in the samples and thereafter
(c) detecting on the carrier the analyte separated from the interfering substance,
wherein the interfering substance is an antibody.

2. The method of claim 1, wherein the sample is a body fluid selected from blood, serum or a body surface fluid.

3. The method of claim 2, wherein the body fluid is selected from mucous membrane fluids or secretions from glands.

4. The method of claim 3, wherein the body fluid is an eye fluid, sweat or saliva.

5. The method of claim 1, wherein the analyte is detected by an immune reaction.

6. The method of claim 1, wherein the analyte is a pathogen or a plurality of pathogens.

7. The method of claim 6, wherein the analyte is a pathogen or a plurality of pathogens associated with conjunctivitis.

8. The method of claim 7, wherein the pathogen is selected from the group consisting of adenoviruses, herpesviruses, chlamydiae, cytomegaloviruses, pseudomonas, streptococci, haemophilus, staphylococci, amobae and combinations thereof.

9. The method of claim 1, wherein the analyte is a low-molecular-weight compound.

10. The method of claim 9, wherein the low-molecular-weight compound is a drug molecule.

11. The method of claim 1, wherein the interfering substance is captured, and thereby separated from the analyte, by an immune reaction.

12. The method of claim 11, wherein the immune reaction comprises immobilizing the interfering substance on the carrier.

13. (canceled)

14. The method of claim 1, wherein the antibody is a human anti-mouse antibody (HAMA), a heterophilic antibody, a rheumatoid factor (RF) or any combination thereof.

15. The method of claim 14, wherein the antibody is the human anti-mouse antibody (HAMA) which is separated from the analyte by an immune reaction with a monoclonal or a polyclonal mouse antibody.

16. (canceled)

17. (canceled)

18. (canceled)

19. (canceled)

20. The method of claim 1, wherein the carrier comprises:
(a) an application zone for applying the sample to the carrier,
(b) a reagent zone containing reagents for detecting the analyte,
(c) a capturing zone for separating the interfering substance from the sample, and
(d) a detection zone for detecting the analyte.

21. The method of claim 20, wherein the sample is directly applied to the application zone.

22. The method of claim 20, wherein the sample is collected with a wiping element from which the sample is transferred, optionally after moistening, to the application zone.

23. The method of claim 20, wherein the capturing zone is located between the reagent zone and the detection zone.

24. The method of claim 20, wherein the capturing zone is located between the application zone and the reagent zone.

25. The method of claim 20, wherein the capturing zone comprises an immobilized capturing reagent which specifically binds to the interfering substance, thereby immobilizing the interfering substance.

26. The method of claim 20, wherein the carrier further comprises a waste zone.

27. The method of claim 1, wherein the carrier is a chromatographic test strip.

28. A test strip for detecting an analyte in a sample which may also contain an interfering substance, comprising:
(a) application zone means for the sample to be applied to the test strip,
(b) a reagent zone containing reagents suitable for detecting the analyte,
(c) capturing zone means for separating on the test strip an interfering substance from the sample,
(d) detection zone means for detecting the analyte on the test strip, and
(e) optionally a waste zone.

29. A method for reducing interference in a method for detecting on a chromatographic carrier an analyte in a sample which may also contain an interfering substance, comprising the steps:
(a) applying the sample to the carrier,
(b) separating the analyte from an interfering substance by passing the sample over a capturing zone located on the carrier to capture the interfering substance, and after step (b)
(c) passing the analyte to a detection zone located on the carrier and detecting the analyte in the detection zone,
wherein the interfering substance is an antibody.

30. The method of claim 29, further including the step of contacting the analyte with at least one detection reagent before the analyte reaches the detection zone.
